(19) United States
(12) Patent Application Publication
(10) Pub. No.: US 2007/0128299 A1
(43) Pub. Date: Jun. 7, 2007

(54) Functional beverage and composition

(75) Inventors: Hiroshi Nagai, Ibaraki (JP); Masaaki Yasue, Ibaraki (JP); Mari Yamamoto, Shizuoka (JP)

Correspondence Address: THELEN REID BROWN RAYSMAN & STEINER LLP, P. O. BOX 640640, SAN JOSE, CA, 95164-0640, US

(73) Assignee: ASAHI SOFT DRINKS CO., LTD., TOKYO (JP)

(21) Appl. No.: 10/588,428

(22) PCT Filed: Feb. 4, 2005
(86) PCT No.: PCT/JP05/01700

§ 371 Date: Aug. 4, 2006

(30) Foreign Application Priority Data
Feb. 6, 2004 (JP) 2004-030490

(51) Int. Cl. A61K 36/82; A61K 31/353

(52) U.S. Cl. 424/729; 514/456

(57) ABSTRACT

Functional beverages and compositions are provided that suppress allergic rhinitis and prevent and treat hyperlipemia and related diseases thereof i.e. arteriosclerosis, obesity and liver diseases.
The O-methylated catechin expressed by the chemical formula (1) shown below is included in a constitutive effective amount as an allergic rhinitis-suppressing drug and a lipid-lowering drug; 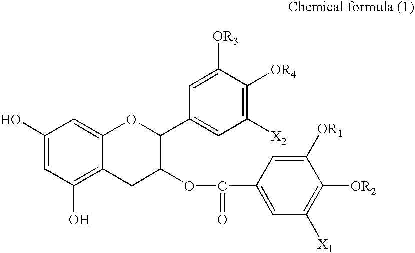 [in which R1, R2, R3 and R4 are each independently a hydrogen atom or a methyl group, X1 and X2 are each independently a hydrogen atom or a hydroxy group.]

### TECHNICAL FIELD

The present invention relates to functional beverages and compositions containing a component that is effective to suppress allergic symptoms, and to prevent and to treat hyperlipemia and the related diseases i.e. arteriosclerosis, obesity, gallbladder/liver diseases and the like.

### BACKGROUND ART

Allergies, which are a type of excessive immunoreaction, develop by action of causal substances, so-called “allergens” such as plants, animals, microorganisms, foods, chemicals etc. Allergic rhinitis in particular, that is an upper respiratory allergic disease represented by pollen, causes such symptoms as paroxysmal sneeze, aqueous nasal drip and nasal obstruction. It is estimated that there exist exclusively thirteen million of pollinosis patients in Japan, which is a serious social problem. In order to suppress such allergic symptoms, it is necessary to inhibit release from mast cells of chemical transmission substances such as histamine, which are capable of inducing the allergic symptoms. A variety of antiallergic drugs have been developed previously that can inhibit histamine release.

For example, an antiallergic drug has been disclosed that contains an extract from oolong tea as an effective component to suppress allergies such as atopic dermatitis; a prophylactic drug and a therapeutic drug for allergies to cedar pollen have been disclosed that are formulated by compounding an oil squeezed from ceder with an antihistamine etc. (see Patent Documents 1 and 2). A supplement drink for pollinosis is also disclosed in which herbs are mixed with Japanese tea (see Patent Documents 3).

In recent years, lifestyle-related diseases including hyperlipemia, diabetes, hypertension and obesity have been increasing year by year along with the westernization of the eating habit. For example, it is estimated in Japan that two-thirds of fatalities are caused by these lifestyle-related diseases, which has become a serious social problem. Hyperlipemia, among these lifestyle diseases, is a disease in which the triglyceride or total cholesterol value of the blood raises due to from various causes such as highly calorified diets, hypokinetic, lowered basal metabolism etc.; it has been found that the elevation of the triglyceride value in particular tends to trigger a variety of serious diseases i.e. related or derived diseases thereof.

The elevation of triglyceride value promotes hypertrophy of fat cells, accumulation of triglyceride within liver cells, and an increase of arteriosclerosis-causing lipoprotein. Then the hypertrophy of fat cells or the accumulation of triglyceride within liver cells raises the risk of developing hepatitis or hepatic cirrhosis through obesity or fatty liver respectively. In addition, since remnant-like lipoprotein cholesterol and small-particle LDL-cholesterol, referred to as arteriosclerosis-causing lipoprotein, are likely to be incorporated into macrophages or vascular endothelial cells, an increase in the blood concentration thereof also raises the risk of developing arteriosclerosis.

That is, control of the triglyceride value in blood within a normal range may lead to suppressing the onset of hyperlipemia-related diseases i.e. arteriosclerosis, obesity, liver diseases etc. For this reason, a variety of drugs and foods etc. have been developed heretofore that may prevent a hypertriglyceridemia.

It has been shown, for example, that catechins in tea leaves may provide various effects such as antioxidant effects, arteriosclerosis suppressing effect, blood-pressure elevation suppressing effect, blood-glucose elevation suppressing effects etc.; therefore, processes for preventing obesity are presented that utilize powders etc. of tea leaves as a raw material of healthy foods so as to raise the basal metabolism and to promote fat-burning, thereby to prevent obesity (see Non-Patent Document 1).
Patent Document 1: Japanese Unexamined Patent Publication No. 10-175874
Patent Document 2: Japanese Unexamined Patent Publication No. 2002-234846
Patent Document 3: Japanese Unexamined Patent Publication No. 2001-348339
Non-Patent Document 1: edited by Kei-ichiro Muramatsu “Cha no Kagaku” published by Asakura Publishing Co., Ltd. (2000)

However, the extract described in Patent Document 1 cannot suppress the symptoms of nasal inflammation; the prophylactic and therapeutic drug described in Patent Document 2 is not effective when cedar pollen has entered into the body. Furthermore, a prophylactic and therapeutic drug compounded with an antihistamine may induce side-effects such as drowsiness, and thus is inappropriate for daily administration. Further, the supplement drink for pollinosis described in Patent Document 3 contains herbs which has a distinctive flavor, thus may not be accepted by everybody.

In addition, it was previously known that catechins are effective to promote fat-burning and to prevent obesity as described above. However, no drug or food or beverage has been proposed that may provide both effects of suppressing allergic symptoms and preventing or treating hyperlipemia and related diseases. Since allergic symptoms and hyperlipemia are definitely different diseases from other, it was difficult to conceive that there would be a substance effective for both of these diseases. Furthermore, the chemical components in tea leaves are different depending on the tea breeds, and there is a number of different tea breeds. Since catechins have many isomers in particular, it has been difficult to investigate which catechin is effective for respective diseases.

The present invention has been accomplished in light of the grounds described above; an object of the present invention is to provide functional beverages and compositions that may suppress allergic rhinitis and else to prevent or treat hyperlipemia and related diseases thereof.

### DISCLOSURE OF THE INVENTION

In order to attain the object described above, the present inventors have investigated diligently and found that a catechin component which found in a kind of tea leaf, may suppress allergic rhinitis as well and prevent or treat hyperlipemia and related diseases thereof; consequently, the present invention has been completed as described in the following.

More specifically, the present invention provides described in the following.

(1) A functional beverage containing an O-methylated catechin expressed by the chemical formula (1) shown below, wherein the functional beverage contains the O-methylated catechin in a quantity of active ingredient as an allergic rhinitis-suppressing drug, a lipid-lowering drug and a gallbladder/liver function-improving drug; 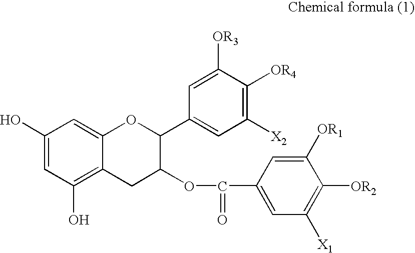
[in which R1, R2, R3 and R4 are each independently a hydrogen atom or a methyl group, X1 and X2 are each independently a hydrogen atom or a hydroxy group.]

In accordance with the invention (1), the quantity of active ingredient of the O-methylated catechin contained in the beverage may make possible to suppress allergic rhinitis and to prevent or to treat hyperlipemia and related diseases thereof. As described above, the catechins may provide various effects such as the effect of an antioxidant, the effect of suppressing arteriosclerosis, the effect of suppressing blood-pressure elevation, the effect of suppressing blood-glucose elevation, sterilization effects, antibacterial effects, deodorized effect etc.

The O-methylated catechins expressed by the chemical formula (1) among others may be excellent in terms of antiallergic effect and triglyceride reduction. The reduction of triglyceride may inhibit the biosynthesis of remnant-like lipoprotein cholesterol and small-particle LDL-cholesterol, which leads to the prevention of arteriosclerosis, and also it may suppress triglyceride accumulation in fat cells, which lowers the risk to develop obesity.

In addition, elevation of the triglyceride value in serum raises the risk of fatty liver and further the risk of hepatitis or liver cirrhosis, since triglyceride is incorporated into liver cells, being decomposed into fatty acid and glycerin by liver lipase; however, the triglyceride value may be lowered in accordance with the invention (1) by virtue of the quantity of active ingredient of the O-methylated catechin within beverages, which may make it possible to prevent these gallbladder/liver dysfunctions.

The term “allergy” as used herein indicates exaggerated immunoreactions in which antibodies for attacking foreign bodies are of produced in excess, and effect healthy cells. Allergies are classified into several types. For overreactions due to IgE antibodies, for example, IgE antibodies that are reactive to specific allergens such as pollens, proteins, mites and house dust, are produced in excess and attach to the surface of mast cells. Then the allergens reach this sites again and the antibodies crosslinking, which leads to the activation of the mast cells thereby to yield inflammatory chemical mediator substances such as histamine, leukotriene etc., consequently causing pollinosis, atopic dermatitis, nettle rash, asthma etc. Here “pollinosis” refers to a symptom where inflammations etc. occur at mucosa of the eyes and nose etc.

Herein, “allergic rhinitis” is a kind of allergic diseases, where antibodies generate upon taking or contacting certain substances, then re-taking or re-contacting the same substance causes a diseased condition due to an antigen-antibody reaction, and it specifically refers to an inflammation at the nasal mucosa that generates an allergic reaction against antigens such as pollens and house dust. This condition is represented by pollinosis due to pollen of ceder and Japanese cypress, and by full-year allergies due to mites and house dust.

The term “allergic rhinitis-suppressing drug” refers to those providing an effect of suppressing the symptoms of the allergic rhinitis described above. The term lipid-lowering drug, anti-obesity drug and liver disease-treating drug in the present invention refer to those providing effects to prevent or treat hyperlipemia, obesity and the liver diseases respectively. In accordance with the present invention, these effects arise by virtue of the inventive O-methylated catechin, thus the inventive O-methylated catechin corresponds to the “allergic rhinitis-suppressing drug”, “lipid-lowering drug” and “gallbladder/liver function-improving drug”. In addition, the inventive beverages refer to those providing these effects simultaneously.

The term “hyperlipemia” refers to the diseases wherein triglyceride or cholesterol exceeds a reference level; and related diseases thereof correspond to arteriosclerosis, obesity and liver diseases including fatty liver. It has been found that obesity may also induce lifestyle-related diseases such as diabetes and hypertension.

The term “constitutive effective amount” refers to an amount where the effective component for suppressing the allergic symptoms and triglyceride value is recognized to sufficiently show the intended effect. Specifically, the O-methylated catechin is included in an amount of 1 mg to 30 mg per 100 ml of beverages.

(2) The functional beverage according to (1), wherein the functional beverage is produced by extracting tea leaves.

In accordance with the invention (2), those produced by the way of extracting tea leaves may easily able to suppress allergic rhinitis and prevent lifestyle-related diseases such as hyperlipemia, obesity and gallbladder/liver diseases by the daily action of drinking tea. The beverages according to the invention (2) may be those provided by filling the extracts into cans, PET bottles etc.

(3) The functional beverage according to (1) or (2), wherein the functional beverage contains the O-methylated catechin in an amount of 1 mg to 30 mg per 100 ml of the beverage.

In accordance with the invention (3), defining the O-methylated catechin amount per 100 ml of the beverage in the range described above may make it possible to provide agreeable beverages with a less bitter taste. When the amount of the O-methylated catechin is above 30 mg, the higher “astringency” is inappropriate for beverages. On the other hand, when the amount is less of than 1 mg, the effect is insufficient.

(4) The functional beverage according to any one of (1) to (3), wherein the O-methylated catechin is derived from one selected from the group consisting of “Benifuuki”, “Benifuji”, “Benihomare”, “Yaeho”, “Surugawase”, “Yutakamidori”, “Kanayamidori”, “Okumusashi”, “Seishin-taipan”, “Seishin-oolong”, “Benibana”, “Benihikari”, “Yamakai”, “Yamamidori”, “Karabeni”, “Koushun”, “Souhuu”, “Okumidori” and any mixtures thereof.

In accordance with the invention (4), the O-methylated catechin is inherently found in the types of tea leaves of “Benifuuki”, “Benifuji”, “Benihomare”, “Yaeho”, “Surugawase”, “Yutakamidori”, “Kanayamidori”, “Okumusashi”, “Seishin-taipan”, “Seishin-oolong”, “Benibana”, “Benihikari”, “Yamakai”, “Yamamidori”, “Karabeni”, “Koushun”, “Souhuu”, “Okumidori” etc., thus employing these tea leaves may make it possible to suppress allergic rhinitis and to inhibit triglyceride. These brands of tea leaves are exemplified of tea leaves containing the O-methylated catechin, but the present invention is not limited to these brands.

(5) The functional beverage according to any one of (1) to (4), wherein the functional beverage is labeled to be effective for at least one of suppressing allergic rhinitis and effective for improving hyperlipemia and gallbladder/liver function.

In accordance with the invention (5), the expression of being effective for at least one of suppressing allergic rhinitis and the expression of being effective for lowering triglyceride may impress consumers with the label of the beverage.

(6) A composition containing an O-methylated catechin expressed by the chemical formula (1), wherein the composition contains the O-methylated catechin in a constitutive effective amount as an allergic rhinitis-suppressing drug and a lipid-lowering drug; 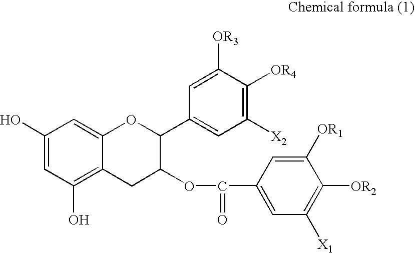
[in which R1, R2, R3 and R4 are each independently a hydrogen atom or a methyl group, X1 and X2 are each independently a hydrogen atom or a hydroxy group.]

In accordance with the invention (6), the inclusion of the O-methylated catechin within the composition in a constitutive effective amount may make it possible to suppress allergic rhinitis and to lower triglyceride. Further, it makes possible to take the composition in a form other than beverages, and various articles may be produced depending on the symptoms, usages and purposes.

The term “composition” as used herein refers to those produced by blending conventional additives with the inventive extracts obtained by extracting tea leaves. The composition encompasses food/beverage products for humans and animals; it may also be a flowable liquid or gel etc. in addition to solids. The composition may be a food; for example, nutritive supplements containing the inventive “allergic rhinitis-suppressing drug” or “lipid-lowering drug” may be provided as foods, or it may take the form of tablets.

(7) The composition according to (6), wherein the composition is a food/beverage, internal medicine, liniment, nose wash, nose drops, cosmetic or eye wash.

In accordance with the invention (7), the O-methylated catechin can be taken more directly due to the composition described above.

The functional beverages according to the present invention may suppress allergic rhinitis without inducing side-effects such as drowsiness, may have a taste which the public kinds favorable, and may suppress triglyceride accumulation along with suppressing allergic rhinitis. Consequently, the daily action of drinking tea may suppress allergic rhinitis easily, and also may prevent lifestyle-related diseases such as hyperlipemia, obesity and gallbladder/liver diseases.

### BRIEF DESCRIPTION OF THE DRAWINGS

FIG. 1 is a graph that shows apparent rhinitis severity calculated from daily reports of the test subjects.

FIG. 2 is a graph that shows the variation of eosinophil count in the nasal discharge of the test subjects.

FIG. 3 is a graph that shows the variation of the triglyceride amount in the blood of the test subjects.

FIG. 4 is a graph that shows the variation of the total bilirubin of the test subjects.

### PREFERRED MODE FOR CARRYING OUT THE INVENTION

The present invention will be explained more specifically below.

Production of Functional Beverage

The functional beverage according to the present invention is one that utilizes the specific O-methylated catechin, derived from tea leaves described below, as the effective component for suppressing allergic rhinitis and as the triglyceride-lowering drug. Drinking the tea produced by steeping the tea leaves in hot water may lead to suppressing allergic rhinitis and lowering triglyceride.

The “O-methylated catechin” according to the present invention, expressed by the chemical formula (1), refers to the O-methylated catechin which is an essential component in purification. Preferably, the O-methylated catechin according to the present invention contains mainly epigallocatechin-3-O-(3-O-methyl)gallate (hereinafter referred to as EGCG3″Me), epicatechin-3-O-(3-O-methyl)gallate (hereinafter referred to as ECG3″Me), epicatechin-3-O-(4-O -methyl)gallate (hereinafter referred to as ECG4″Me), epigallocatechin-3-O-(4-O-methyl)gallate (hereinafter referred to as EGCG4″Me), gallocatechin-3-O-(3-O-methyl)gallate (hereinafter referred to as GCG3″Me), catechin-3-O-(3-O-methyl)gallate (hereinafter referred to as CG3″Me), catechin-3-O-(4-O-methyl)gallate (hereinafter referred to as CG4″Me), or gallocatechin-3-O-(4-O-methyl)gallate (hereinafter referred to as GCG4″Me) and isomers thereof. The general effect of the O-methylated catechin is to inhibit the release of inflammatory substances such as histamine of chemical mediator, and to suppress type-I and type-IV allergies. 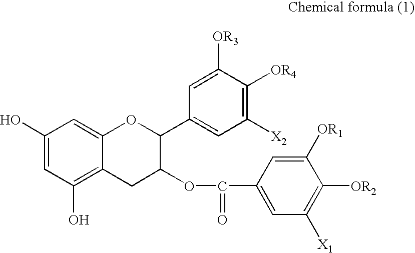
[in which R1, R2, R3 and R4 are each independently a hydrogen atom or a methyl group, X1 and X2 are each independently a hydrogen atom or a hydroxy group.]

Preferably, the amount of the EGCG3″Me, ECG3″Me, ECG4″Me, EGCG4″Me, GCG3″Me, CG3″Me, CG4″Me or GCG4″Me is 1 to 30 mg, more preferably 2 to 20 mg, still more preferably 5 to 15 mg per 100 ml of the beverage. When the amount is less than 1 mg, the effects to suppress the allergic rhinitis and to lower the triglyceride are decreased. When the amount is more than 30 mg, the higher “astringency” makes it inappropriate for beverages. If the “astringency” can be eliminated by certain means, the amount may be more than 30 mg in some cases. The amount may also be suitably adjusted depending on the kind of the O-methylated catechin.

The O-methylated catechin according to the present invention may be derived from designated tea leaves. Examples of the tea leaves, containing the O-methylated catechin, include “Benifuuki”, “Benifuji”, “Benihomare”, “Yaeho”, “Surugawase”, “Yutakamidori”, “Kanayamidori”, “Okumusashi”, “Seishin-taipan”, “Seishin-oolong”, “Ohba-oolong”, “Hououtansou”, “Houousuisen”, “Hakuyoutansousuisen”, “Kohshikou”, “Buisuisen”, “Benibana”, “Benihikari”, “Yamakai”, “Yamamidori”, “Karabeni”, “Koushun”, “Okumidori” and mixtures thereof. These tea leaves may be used alone or mixtures of two or more.

In order to make the O-methylated catechin described above sufficiently effective to suppress allergic rhinitis and onset of antilipemic action, additives may be compounded, alone or in combination thereof, to the functional beverages according to the present invention, such as antioxidants, flavors, various esters, organic acids, organic acid salts, inorganic acids, inorganic acid salts, inorganic salts, dyes, emulsifiers, preservatives, condiments, sweeteners, acidifiers, juice extracts, vegetable extracts, nectar extracts, pH adjusters, quality stabilizer etc.

Examples of the sweeteners include sugar, glucose, fruit sugar, isomerized sugar syrup, glycyrrhizin, stevia, aspartame, fructo oligosaccharides, galacto oligosaccharides etc. Examples of the acidifiers include juices extracted from natural products, and also citric acid, tartaric acid, malic acid, lactic acid, fumaric acid and phosphoric acid. It is preferred that citric acid or malic acid is added to beverages in a content of 0.1 to 5 g/L, preferably 0.5 to 2 g/L. Examples of the antioxidants include L-ascorbic acid, sodium L-ascorbate, erythorbic acid, sodium erythorbate etc. It is preferred that the content is 0.005 to 0.5 weight %, preferably 0.01 to 0.1 weight % in the beverages.

The containers utilized for the functional beverages according to the present invention may be provided, similarly as conventional beverages, in usual forms such as shaped-containers based on polyethylene terephthalate (so-called PET bottles), metal cans, paper containers combined with metal foils or plastic films, or bottles.

When the containers can be subjected to appertization after filling the containers as in the case of metal cans, the containers may be produced under conditions determined by food hygiene laws. As for PET bottles or paper containers, which cannot be retort-sterilized, processes may be employed wherein the containers are sterilized in advance at a high temperature for a short period by use of a plate-type heat exchanger, for example, under similar sterilizing conditions as above described, and then being cooled to a certain temperature, thereafter the containers being filled. Further, previously filled containers may be compounded and filled with another component under sterile conditions. In addition, it is able to heat and sterilize under acidic conditions then returning the pH to neutral under sterility. Also, it is able to heat and sterilize under neutral conditions and then returning the pH to acidic under sterility.

Preparation of Composition

The compositions according to the present invention are those in which the O-methylated catechin component acts as an effective component for suppressing allergic rhinitis and also as a triglyceride-lowering drug. The compositions may be produced through extracting O-methylated catechin from tea leaves described above by conventional processes. The temperature at the extraction process does not have to be definitely specified, as long as it is higher than the melting temperature and lower than the boiling temperature of the solvent, preferably 10 to 100° C. in the case of water and 10 to 40° C. in the case of methanol. The extracting period is preferably from 10 seconds to 24 hours.

Preferably, dried tea leaves are processed into a powder by a pulverizing or milling process etc., then an extraction solvent is added to the powder, and the extract or its treated product is utilized. Examples of the extraction solvents include water; lower alcohols such as methanol, ethanol, propanol, isopropanol, butanol and isobutanol; ethers such as ethylether and dioxane; ketones such as acetone; water; an ethanol or water-ethanol mixed solvent are preferred.

The resulting extracts may be used without subsequent processing, for use of the compositions according to the present invention; preferably, conventional processes for chemical separation-purification are employed. For example, liquid-liquid distribution, thin-layer chromatography, absorption-column chromatography, distribution-column chromatography, gel-filtration column chromatography, ion-exchange column chromatography, electrophoresis, or high-performance liquid chromatography may be employed. These separation-purification means may be combined as appropriate.

The compositions according to the present invention may be utilized for various applications such as pharmaceuticals and foods. As for pharmaceuticals, they may be provided for treating allergic rhinitis, hyperlipemia, obesity and gallbladder/liver diseases. As for the foods, they may be compounded as food additives into specified health foods, special nutritious foods, dietary supplements, health foods etc. They may be added to a variety of foods. As for beverages, they may be compounded with beverages as specified health foods, special nutritious foods or dietary supplements; other energy drinks, healthy drinks or various healthy teas; and other drinks. As for the other foods, confectioneries, breads, noodles, soybeans processed products, dairy products, egg processed products, fish cakes, and condiments etc. may be given as examples. As for cosmetics, the compositions according to the present invention may be added to skin-care, foundation or make-up products for the purpose of alleviating or preventing pollinosis symptoms or sliming.

As for phanmaceuticals, the compositions according to the present invention may be orally administered without modification or with diluting by use of water etc; alternatively they may be formulated with conventional medical carriers. For example, they may be administered in the form of oral liquid formlations such as syrups, or in the form of oral solid formulations such as tablets, capsules, granules and powders, through processing into an essence or powder and then compounding with pharmaceutically acceptable carriers. The pharmaceutically acceptable carriers may be various organic or inorganic carrier substances utilized conventionally for formulation materials, and are compounded as an excipient, lubricant, binder or disintegrating agent in solid formulations, or a solvent, excipient, suspending agent or binder in liquid formulations. Also fonnulation additives such as preservatives, antioxidants, colorants and sweetening agents may be employed if necessary. In addition, nose washers, nose drops, or eye washes may be produced by use of conventional pharmaceutical carriers.

### EXAMPLES

### Example 1

### Investigation of Allergic Rhinitis-Suppressing Effects

“Benifuuki” tea leaves were extracted at 90° C. using purified water in an amount 30 times of the tea leaves to produce an extracted liquid, then a sodium bicarbonate water conditioner and vitamin C were added and mixed. The mixture was sterilized and filled into a sealed container (a 250 ml paper pack in this example) under nitrogen to prepare Test Beverage 1.

### Comparative Example 1

As an example for comparison, “Yabukita” tea leaves were measured up by purified water such that the contents of the components other than the O-methylated catechin were the same as those of Test Beverage 1, then a sodium bicarbonate water conditioner and vitamin C were added and mixed. The mixture liquid was sterilized and filled into a sealed container under nitrogen to prepare Test Beverage 2.

### Comparative Example 2

As a comparative example similar to Comparative Example 1, “barley tea” was measured up by purified water such that the contents of the components other than the O-methylated catechin were the same as those of Test Beverage 1, then a sodium bicarbonate water conditioner and vitamin C were added and mixed. The mixture liquid was sterilized and filled into a sealed container under nitrogen to prepare Test Beverage 3.

The contents of O-methylated catechins within Test Beverages 1 to 3 are shown in Table 1.

TABLE 1

| | Test | | |
|---|---|---|---|
| (mg/100 ml) | Beverage 1 | Test Beverage 2 | Test Beverage 3 |
| Total Content of | 8.1 | 0 | 0 |
| O-Methylated | | | |
| Catechins | | | |
| Total Content of | 104.8 | 109.2 | 0 |
| Catechins | | | |

### Example 2

### Investigation of Allergic Rhinitis-Suppressing Effect

The test beverages prepared in Example 1 and Comparative Examples 1 to 2 were evaluated with respect to the suppression of pollinosis. The test was carried out such that perennial rhinitis patients were divided into groups of 20 to 23 persons each, and the respective groups were made to take the Test Beverages 1 to 3 for 12 weeks, then the effects on the rhinitis were examined. The test was delegated to Sougouikagakukenkyu-jyo Co., Ltd., and the test protocol was designed in collaboration with Sougouikagakukenkyu-jyo Co., Ltd., Asahi Soft Drinks Co., Ltd., Asahi Breweries Ltd. and the National Agriculture Research Center.

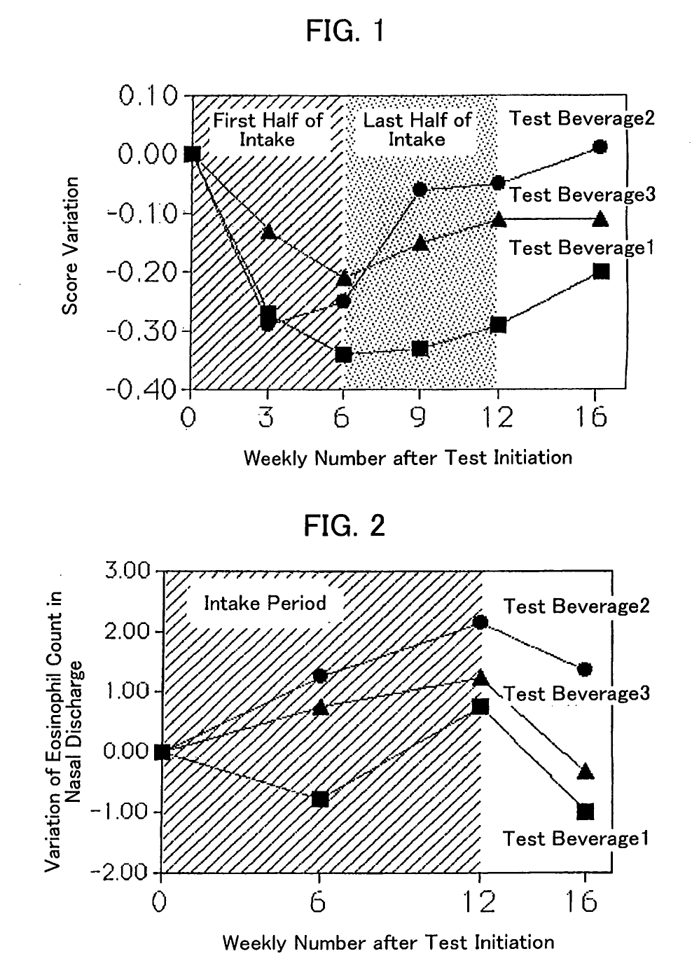

The experimental subjects were directed to drink two bottles of the test beverage 1, 2 or 3, each contained in a container of 250 ml, per day (500 ml/day). The experimental subjects were made to prepare a daily report every day from two weeks before the initial drinking till four weeks after the last drinking i.e. the sixteenth week, thereby severity scores were calculated in accordance with the diagnosing standard of the Japanese Society of Allergology. The test was carried out under double-blind conditions, i.e. neither the experimental subjects nor the doctors in attendance knew the test groups. FIG. 1 shows the results of the apparent severity (nasal symptomatic score) calculated from averaging the values every three weeks. In the figure, the box marks indicate Test Beverage 1, circle marks indicate Test Beverage 2 and triangle marks indicate Test Beverage 3.

In the two control groups of Test Beverages 2 and 3, the symptoms reappeared after the sixth week subsequent to the initial drinking, whereas the symptoms remained mitigated were in the group of Test Beverage 1.

Further, the eosinophil count in nasal discharge was ascertained to decrease. From the results described above, it was demonstrated that “Benifuuki” is effective in that the allergy-mitigating effect of “Benifuuki” exceeds those of “barley tea” and “Yabukita”. The results are shown in FIG. 2, in which the box marks indicate Test Beverage 1, circle marks indicate Test Beverage 2 and triangle marks indicate Test Beverage 3 in the same manner as in FIG. 1. As a result it is possible to provide beverages which are confirmed to be effective for allergic rhinitis in humans.

### Example 3

### Investigation of the Triglyceride-Lowering Effect

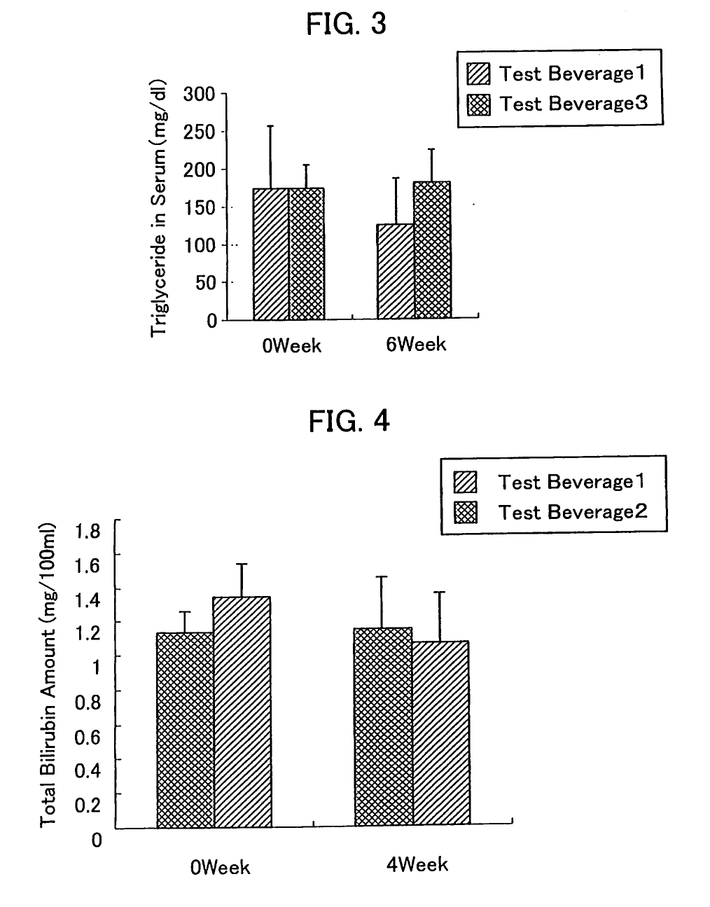

Ten healthy adults, whose fasting triglyceride value was 100 to 350 mg/dL, were divided into two groups of five persons (each group having four men and one woman), and were directed to drink Test Beverage 1 or Test Beverage 3 of Comparative Example 2 for six weeks. Each beverage was contained in a 250 mL bottle, and the subjects were made to drink two bottles per day (500 ml/day). The triglyceride values (average±SD value) were determined after fasting overnight at the test initiation and at the last drinking. The results are shown in Table 2 and FIG. 3. These results demonstrate that the intake of Test Beverage 3 containing no O-methylated catechin provided no effect on the triglyceride values in serum, whereas Test Beverage 1 containing O-methylated catechin significantly lowered the triglyceride values.

TABLE 2

| | 0 week | Sixth Week |
|---|---|---|
| Test Beverage 1 | 174 ± 82 | 125 ± 62 |
| (Benifuuki) | | |
| Test Beverage 3 | 174 ± 31 | 181 ± 42 |
| (barley tea) | | |

p < 0.05 vs 0 week

### Example 4

### Investigation of the Gallbladder/Liver Function-Correcting Effect

Nine perennial allergic rhinitis patients, whose total bilirubin value being 1.1, were divided into one group of five persons (Benifuuki) and one group of four persons (placebo: Yabukita), and were directed to drink the Test Beverage 1 of Example 1 and Test Beverage 2 of Comparative Example 1 for four weeks. Each beverage was contained in a bottle of 250 mL, and the subjects were made to drink six bottles per day (1500 ml/day). The total bilirubin values (average±SD value) were determined after fasting overnight at the test initiation and at the last drinking. The results are shown in Table 3 and FIG. 4. From these results, it was confirmed that such effects may be taken as a lipid-lowering drug and a gallbladder/liver function-improving drug.

TABLE 3

| | 0 week | Sixth Week |
|---|---|---|
| Test Beverage 1 | 1.34 ± 0.20 | 1.06 ± 0.29 |
| (Benifuuki) | | |
| Test Beverage 2 | 1.13 ± 0.12 | 1.15 ± 0.3 |
| (Yabukita) | | |

p < 0.05 vs 0 week

1. A functional beverage containing an O-methylated catechin expressed by the chemical formula (1) shown below, wherein the functional beverage contains the O-methylated catechin in a quantity of active ingredient as an allergic rhinitis-suppressing drug, a lipid-lowering drug and a gallbladder/liver function-improving drug. 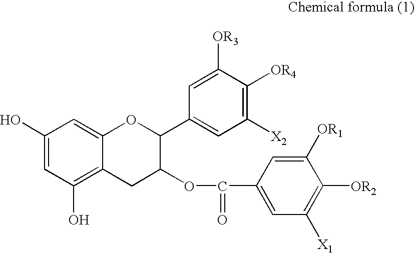
[in which R1, R2, R3 and R4 are each independently a hydrogen atom or a methyl group, X1 and X2 are each independently a hydrogen atom or a hydroxy group.]

2. The functional beverage according to claim 1, wherein the functional beverage is produced by extracting tea leaves.

3. The functional beverage according to claim 1, wherein the functional beverage contains the O-methylated catechin in an amount of 1 mg to 30 mg per 100 ml of the beverage.

4. The functional beverage according to claim 1, wherein the O-methylated catechin is derived from one selected from the group consisting of “Benifuuki”, “Benifuji”, “Benihomare”, “Yaeho”, “Surugawase”, “Yutakamidori”, “Kanayamidori”, “Okumusashi”, “Sei shin-taipan”, “Seishin-oolong”, “Ohba-oolong”, “Benibana”, “Benihikari”, “Yamakai”, “Yamamidori”, “Karabeni”, “Koushun”, “Souhuu”, “Okumidori” and mixtures thereof.

5. The functional beverage according to claim 1, wherein the functional beverage is labeled to be effective for at least one of suppressing allergic rhinitis and effective for improving hyperlipemia and gallbladder/liver function.

6. A composition containing an O-methylated catechin expressed by the chemical formula (1) shown below, wherein the composition contains the O-methylated catechin in a constitutive effective amount as an allergic rhinitis-suppressing drug and a lipid-lowering drug. 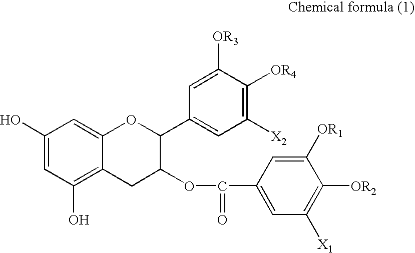
[in which R1, R2, R3 and R4 are each independently a hydrogen atom or a methyl group, X1 and X2 are each independently a hydrogen atom or a hydroxy group.]

7. The composition according to claim 6, wherein the composition is a food/beverage, internal medicine, liniment, nose wash, nose drops, cosmetic or eye wash.
